(19) United States
(12) Patent Application Publication
(10) Pub. No.: US 2017/0049390 A1
(43) Pub. Date: Feb. 23, 2017

(54) BODY PARAMETER DETECTING METHOD AND BODY PARAMETER DETECTING DEVICE

(71) Applicant: BOE TECHNOLOGY GROUP CO., LTD., Beijing (CN)

(72) Inventors: Lijie Zhang, Beijing (CN); Zhenglong Li, Beijing (CN)

(21) Appl. No.: 15/155,716

(22) Filed: May 16, 2016

(30) Foreign Application Priority Data
Aug. 20, 2015 (CN) 201510516304.8

(51) Int. Cl. A61B 5/00; A61B 5/053; A61B 8/14; A61B 8/08

(52) U.S. Cl. CPC A61B 5/4872; A61B 8/0858; A61B 5/0537; A61B 5/7275; A61B 8/14; A61B 8/5223

(57) ABSTRACT

A body parameter detecting device includes: a detection module and a determination module. The detection module is configured to detect fat thickness and fat density of sites to be detected of a human body. The determination module is configured to determine body parameters at least based on the fat thickness and the fat density. This device has simple operations for detection steps and low price, and is suitable for common people users. Also, a body parameter detecting method is provided.

### TECHNICAL FIELD

Embodiments of the present disclosure relate to the field of sensor technology, in particular to a human body parameter detecting method and a human body parameter detecting device.

### BACKGROUND

More and more attention has been paid to body weight due to the requirements for somatotype and health. Many factors contribute to weight, weight reduction aiming at reducing weight tends to cause significant deviation in the reduction effect. Aiming at reducing weight merely usually results in some quick ways to lose weight being used. However these methods generally reduce muscles and moisture, and therefore the weight is likely to increase again.

A human body mainly consists of skeleton, internal organs, thin muscle tissues, moisture and fat. The skeleton and internal organs are basically constant, while muscles are main sites for heat consumption. The reduction of muscle would lead to decrease of metabolism. Therefore muscles can not be reduced in weight reduction. It is easy to expel and supplement moisture. While fat not only has extreme influence on one's somatotype, but also drastically damages human's health. Therefore, the fundamental goal of weight reduction is to reduce fat.

However, obesity does not mean excessive weight exclusively, but means abnormal or excessive fat accumulation. To determine whether one is obese, it is possible to measure the proportion of fat tissue in the body, namely the fat percentage.

### SUMMARY

According to one aspect of this disclosure, a body parameter detecting device is provided, which includes a detection module and a determination module. The detection module is configured to detect fat thickness and fat density of sites to be detected of a human body. The determination module is configured to determine body parameters at least based on the fat thickness and the fat density.

According to another aspect of this disclosure, a body parameter detecting method is provided, which includes: detecting fat thickness and fat density of a site to be detected of a human body; determining body parameters according to the fat thickness and the fat density.

### BRIEF DESCRIPTION OF DRAWINGS

The present disclosure will become more fully understood from the detailed description given hereinafter and the accompanying drawings used when describing the embodiments or prior art, which are given by way of illustration only, and thus are not limitative of the present disclosure. Those skilled in the art can obtain other drawings according to the drawings shown herein without inventive labors. Wherein:

FIGS. 1-4 are structure diagrams of an body parameter detecting device provided in embodiments of the present disclosure respectively;

FIG. 5 is flow chart of a body parameter detecting method provided in embodiments of the present disclosure.

### DETAILED DESCRIPTION

Thereafter will describe the solutions according to the embodiments of the present disclosure clearly and fully in connection with the drawings in the embodiments of the present disclosure. Obviously, the embodiments described are only a part of embodiments of the present disclosure instead of all of the embodiments. All of other embodiments educing from the embodiments of the present disclosure by those ordinary skilled in the art without any inventive labors are fallen into the scope of the present disclosure.

A variety of equipments and methods may be used to detect body fat, such as underwater weighing measurement, which has small error and high precision, but requires dedicated test space and tools and has many operation steps, and hence only suitable for a lab test rather than one's self testing; skinfold measurement, which so strongly depends on the phantom used in the calculation process, but data of European and American phantom can not be used in Asia, and Japanese phantom can not be used to calculate Chinese data well; dual energy X-ray absorption measuring method, which is a relatively new method with high precision but suffers expensive test costs and long test duration (10-20 minutes per human), and can only be used in advanced labs rather than outside a lab; and near infrared rays measuring method, nuclear magnetic resonance imaging, thin body electrical conductivity, computer controlling (X-ray) tomography, air displacement, and bioelectrical impedance etc., which tend to be limited by one or more of test precision, expensive testing equipments and test arena and can not be applied to common people. Accordingly, it is desired to have a body parameter detecting device with simple detection method and suitable for common people to facilitate a user to detect body parameters of himself or herself and guide a reasonable and health life for the user.

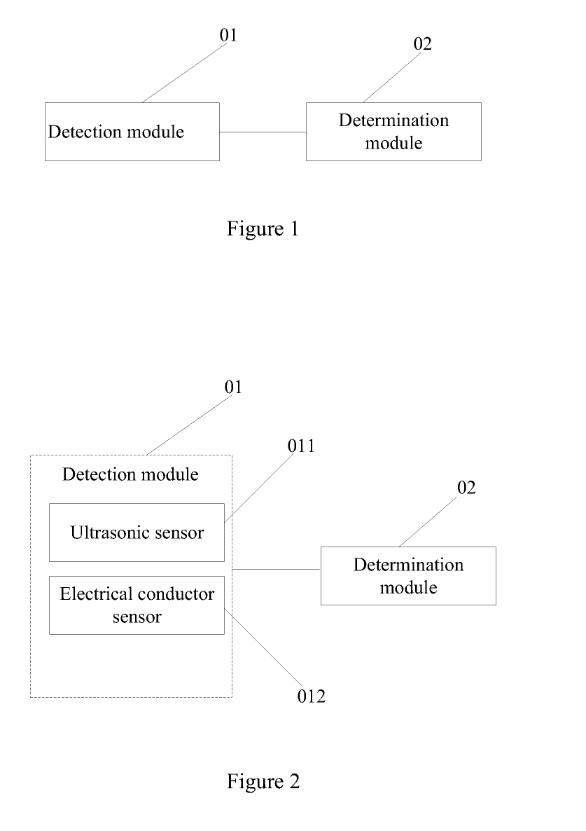

According to embodiments of the present disclosure, there is provided a body parameter detecting device as shown in FIG. 1, including a detection module 01 and a determination module 02. The detection module 01 and the determination module 02 may be implemented by software, hardware, firmware and any combinations thereof, for example.

The detection module 01 is configured to detecting fat thicknesses and fat densities of sites to be detected of a human body.

The determination module 02 is configured to determine body parameters at least based on the fat thickness and the fat density.

Said body parameters may be the human body's physiological parameters such as obesity index and fat status.

The above-described body parameter detecting device provided in embodiments of the present disclosure includes: a detection module 01 configured to detect fat thickness and fat density of a body site to be detected and a determination module 02 configured to determine the obesity index of a body according to the fat thickness and the fat density.

The body parameter detecting device provided in embodiments of the present disclosure has a simple structure and a low price, is suitable for common people users, and may facilitate a user to detect fat thickness and fat density of himself or herself to obtain the corresponding obesity index information.

Furthermore, it is possible to analyze future health tendency according to the obesity index, namely the historical data, to provide the user with a scientific fat detection method, and it is also possible to facilitate the user to correctly consider his or her obesity and health information and reasonably guide the user for healthy exercise and diet. As compared to underwater weighing and skinfold measurement that are limited by test precision, expensive testing equipment and test arena, the body parameter detecting device provided in embodiments of the present disclosure has a simple structure and a low price and is suitable for common people users.

According to another embodiments of the present disclosure, as shown in FIG. 2, in the above-described body parameter detecting device, the detection module 01 may includes an ultrasonic sensor 011 configured to detect fat thickness and an electrical conductor sensor 012 configured to detect fat density. For example, the ultrasonic sensor 011 measures the body sites to be tested ultrasonically to generate a corresponding ultrasonic echo diagram, and determines the fat thickness of the body sites to be tested according to the generated ultrasonic echo diagram. The electrical conductor sensor 012 tests electrical conductivity of the body sites to be detected via positive and negative electrodes to obtain currents through the body sites to be detected and determines fat densities of the body sites to be detected according to the obtained currents.

According to another example of the present disclosure, the ultrasonic sensor 011 detects fat thickness of a human body with ultrasonic waves, for example, the fat thickness on the abdomen of the human body represents the obesity degree of a person to a certain degree. Therefore the ultrasonic measurement result of abdomen may be used as an index for determining obesity degree. Ultrasonic measurement implements measurements primarily using different propagation speed and propagation time of local sound in different media. Tissues and organs of a human body have different waveforms of echograms due to the different tissue acoustic characteristics. Therefore, it is possible to determine the location and thickness of fat according to different reflection rules such as waveform distribution and amplitude.

The electrical conductor sensor 012 may detect fat density with positive and negative electrodes. Since body tissues of different media have different electrical conductivities, it is possible to determine constituent components of a human body tissue by different electrical conductivities. Generally, an electrical conductor sensor has two electrodes, positive and negative electrodes, and when a certain site of a human body is to be measured, they are applied to two ends of the site. A current flowing through the body may be detected by applying a small voltage, and a resistance of the present site may be calculated in turn, thereby obtaining the electrical conductivity of the site. Human tissues are simply classified into conductive body fluids and muscles and non-conductive fat tissue. Upon measurement, the minute current transmitted from electrode plate flows through the body. In case of high fat percentage, the measured bioresistance is large, and vice versa. Therefore, it is possible to determine whether there is much fat at the site.

In summary, fat thickness and fat density are detected by the ultrasonic sensor and the electrical conductor sensor respectively, and it is possible to determine obesity index of the user according to the detected fat thickness and fat density. It is further possible to facilitate the user to properly consider the obesity and health information of himself or herself and reasonably guide the user for healthy exercise and diet and for health weight reduction.

According to one example of the present disclosure, the determination module 02 is configured to: consider a set including fat thickness and fat density as the current parameter set of the site to be detected, and determine the obesity index corresponding to the current parameter set of the site to be detected according to the correspondence between a preset obesity index and a preset parameter set. Wherein, the preset parameter set includes the sample fat thickness and fat density of the site to be detected. For example, the determination module 02 stores in advance the corresponding relationship between the obesity index and the preset parameter set and the parameter set including the sample fat thickness and fat density of the site to be detected. Thus, it is possible to determine the obesity index of the site to be detected by comparing the detected fat thickness and fat density against the pre-stored data sample and correspondence relationship, and thereby facilitate the user to properly consider the obesity characteristics of himself or herself For example, if some people have fat abdomen, detecting the abdomen fat thickness and density allows the users to learn about their obesity information and in turn guide users for healthy exercise and diet, thereby preventing users from reducing weight blindly and impacting their health.

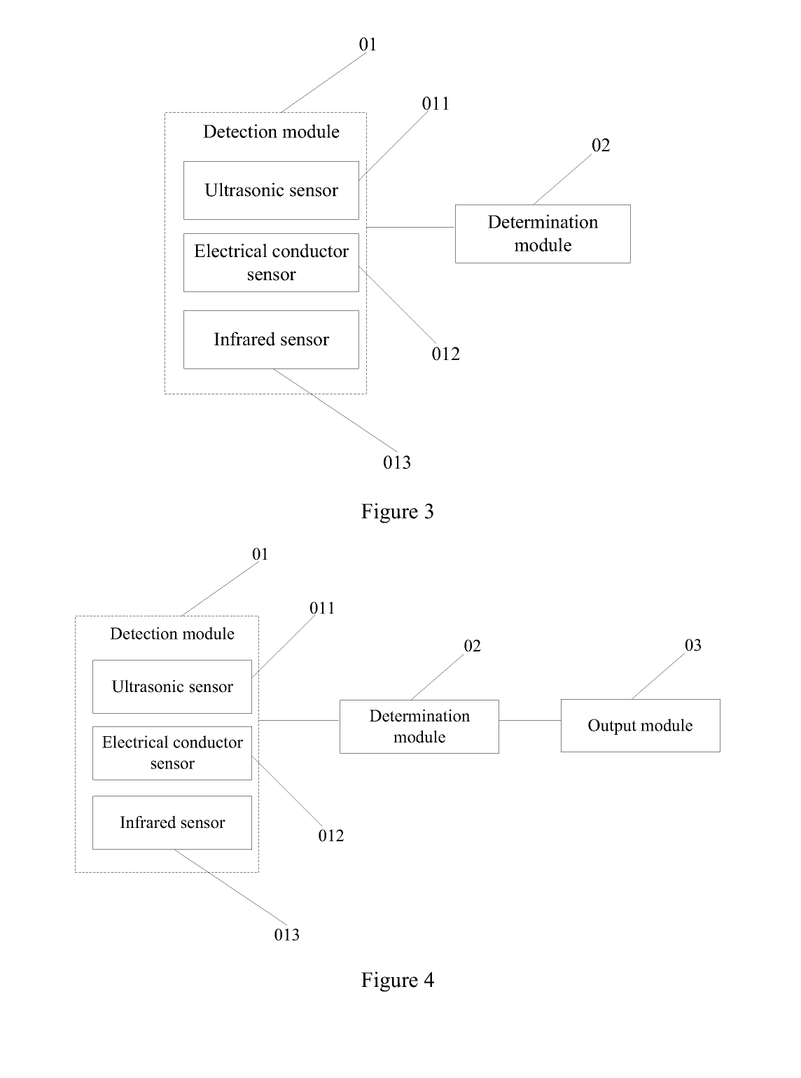

According to yet other embodiments of the present disclosure, as shown in FIG. 3, the detection module 01 may further include an infrared sensor 013. The infrared sensor 013 implements transmission detection on the site to be detected with infrared light, generates a spectral information diagram of the corresponding site to be detected and determines the tissue density of the site to be detected according to the generated spectral information diagram. The determination module 02 can determine obesity index of the corresponding site of a human body according to fat thickness, fat density and tissue density. In the above-mentioned body parameter detecting device provided in the embodiments of the present disclosure, it is possible to detect density of a body tissue, such as fat tissue and muscle tissue, with an infrared sensor. Infrared spectrum of corresponding detected tissue is generated by implementing infrared transmission detection on body's tissues. For example, when fat molecules in the body are irradiated by infrared light, some energy of the light is absorbed. It is possible to obtain a diagram having characteristic information of the different detected tissues such as fat and muscle, containing density information of corresponding different tissues, by measuring the transmission spectra after absorption by different tissues such as fat. Accordingly, based on different tissue densities, it is possible to determine incidence rate of some diseases by medical theoretical analysis, and it is also possible to determine the body's obesity index and health status according to the measured fat density, fat thickness and other tissue densities, thereby obtaining some health information about the body.

According to embodiments of the present disclosure, as shown in FIG. 4, the body parameter detecting device may further includes an output module 03. The output module 03 is configured to output the determined body parameters to the user. In the above-mentioned body parameter detecting device provided in embodiments of the present disclosure, the body parameters such as fat thickness, fat density and tissue density may be output to the user by the output module. Alternatively, the body obesity index and health information determined according to the fat density, fat thickness and tissue density may be output to the user via the output module to facilitate the user to obtain detection results, which in turn allows the user to learn about the obesity information and health condition of himself or herself properly and guides the user for healthy exercise and diet.

Optionally, the output module may be configured to output the determined body parameters to the user via a display module. Alternatively, they are output to the user via a speech output device. In the body parameter detecting device provided in embodiments of the present disclosure, the output module may output the obesity index information and health condition to the user by means of display picture or speech, that is, outputting the determined body parameters to the user via respective display module or speech output device. In this way, the user can obtain respective information more directly and conveniently, and in turn the user can properly learn about the obesity information and health condition of himself or herself and be guided for healthy exercise and diet.

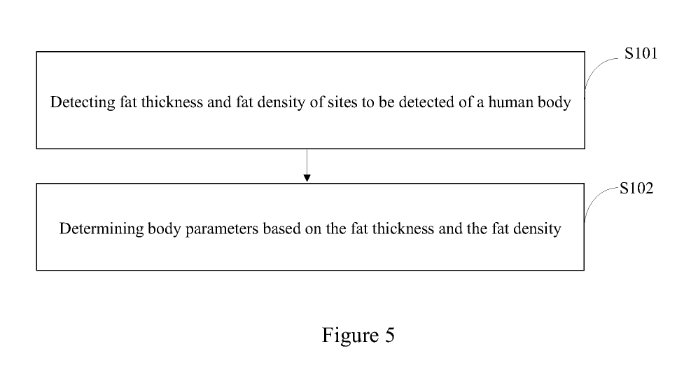

Based on the same inventive concept, embodiments of the present disclosure provides a body parameter detecting method as shown in FIG. 5, which may include the following steps.

In step S101, fat thicknesses and fat densities of sites to be detected of a human body are detected.

In step S102, body parameters are determined according to the fat thickness and the fat density.

In the above-described body parameter detecting method provided in the embodiment of the present disclosure, corresponding data information is obtained by detecting fat thickness and fat density and the user's body parameters such as obesity index are determined according to the obtained data information. And it is possible to analyze future health tendency according to the historical data to provide the user with a scientific fat detection method, and it is also possible to facilitate the user to correctly consider his or her obesity and health information and reasonably guide the user for healthy exercise and diet. As compared to under water weighing and skinfold measurement that are limited by test precision, expensive testing equipment and test arena, the body parameter detecting method provided in embodiments of the present disclosure has simple operations for detection steps and is suitable for common people users. It is possible to facilitate the user to detect fat thickness and density of himself or herself to obtain corresponding obesity index information, and in turn facilitate the user to properly recognize his or her obesity and health information and properly guide the user for healthy exercise and diet and for healthy weight reduction.

In step S101, detecting fat thickness and fat density of the site to be detected may include: detecting fat thickness of the site to be detected by the ultrasonic wave and detecting fat density of the site to be detected by the electrical conductor sensor.

For example, the body sites to be tested are measured ultrasonically to generate a corresponding ultrasonic echo diagram, and the fat thickness of the body sites to be tested is determined according to the generated ultrasonic echo diagram. Electrical conductivity of the body sites to be detected is measured via positive and negative electrodes of the electrical conductor sensor to obtain currents through the body sites to be detected, and fat densities of the body sites to be detected are determined according to the obtained currents. For example, the fat thickness of the body may be detected by ultrasonic waves and the fat density may be detected by electrical conductivity test of positive and negative electrodes. Thus, the detected fat thickness and fat density may provide detection data for determining the user's obesity index and in turn facilitate the user to properly consider obesity and health information of himself or herself It is possible to reasonably guide the user for healthy exercise and diet and for healthy weight reduction.

In step S102, determining body parameters according to fat thickness and fat density may include considering a set including fat thickness and fat density as the current parameter set of said site to be detected, and determining the obesity index corresponding to the current parameter set of the site to be detected according to the correspondence between a preset obesity index and a preset parameter set. The preset parameter set includes the sample fat thickness and fat density of the site to be detected. For example, it is possible to determine the obesity index of the site to be detected by comparing the detected fat thickness and fat density against the pre-stored data sample and correspondence relationship, and in turn facilitate the user to properly consider the obesity characteristics of himself or herself For example, if some people have fat abdomen, detecting the abdomen fat thickness and density allows the users to learn about their obesity information and in turn guide users for healthy exercise and diet, thereby preventing users from reducing weight blindly and impacting their health.

Optionally, the above-described body parameter detecting method provided in embodiments of the present disclosure may further include implementing transmission detection on the site to be detected with infrared light, generating a spectral information diagram of the corresponding site to be detected and determining the tissue density of the site to be detected according to the generated spectral information diagram. Then the obesity index of the corresponding site of a human body is determined according to fat thickness, fat density and tissue density. For example, it is possible to detect density of a body tissue, such as fat tissue and muscle tissue, by infrared light transmission. Infrared spectrum of corresponding detected tissue is generated by implementing infrared transmission detection on body's tissues. For example, when fat molecules in the body are irradiated by infrared light, some energy of the light is absorbed. It is possible to obtain a diagram having characteristic information of the different detected tissues such as fat and muscle, containing density information of corresponding different tissues, by measuring the transmission spectra after absorption by different tissues such as fat. Accordingly, based on different tissue densities, it is possible to determine incidence rate of some diseases by medical theoretical analysis, and it is also possible to determine the body's obesity index and health status according to the measured fat density, fat thickness and other tissue densities, thereby obtaining some health information about the body.

According to embodiments of the present disclosure, the above-mentioned body parameter detecting method may further include outputting the determined body parameters to the user. For example, the detected body parameters such as fat thickness, fat density and tissue density are output to the user in time. It is also possible to output the body obesity index and health information determined according to the fat density, fat thickness and tissue density to the user in time. For example, a transmission instruction is generated corresponding to the determined body parameters, and according to the transmission instruction, the corresponding body parameters are output to the user to facilitate the user to obtain detection results, which in turn allows the user to learn about the obesity information and health condition of himself or herself properly and guides the user for healthy exercise and diet.

Outputting the determined body parameters to the user may include outputting the determined body parameters to the user via a display module. Alternatively, they are output to the user via a speech output device. For example, the obesity index information and the health condition may be output to the user by displayed picture or speech. That is, the determined body parameters are output to the user via respective display module or speech output device. In this way, the user can obtain respective information more directly and conveniently, and in turn the user can properly learn about the obesity information and health condition of himself or herself and be guided for healthy exercise and diet.

Through the above-mentioned embodiments provided in the present disclosure, for example, the detecting device includes a detection module configured to detect fat thickness and fat density of a body site to be detected and a determination module configured to determine the body parameters at least according to the fat thickness and the fat density. In this way, corresponding data information is obtained by detecting fat thickness and fat density and the user's body parameters such as obesity index are determined according to the obtained data information. And it is possible to analyze future health tendency according to the historical data to provide the user with a scientific fat detection method, and it is also possible to facilitate the user to correctly consider his or her obesity and health information and reasonably guide the user for healthy exercise and diet. As compared to devices for under water weighing and skinfold measurement that are limited by test precision, expensive testing equipment and test arena, the body parameter detecting device provided in embodiments of the present disclosure has simple operations for detection steps, and simple device and low price, and is suitable for common people users. It is possible to facilitate the user to detect fat thickness and density of himself or herself to obtain corresponding obesity index information, and in turn facilitate the user to properly recognize his or her obesity and health information and properly guide the user for healthy exercise and diet and for healthy weight reduction.

The present application claims priority of China Patent application No. 201510516304.8 filed on Aug. 20, 2015, the content of which is incorporated in its entirety as part of the present application by reference herein.

What is claimed is:

1. A body parameter detecting device, comprising a detection module and a determination module; wherein
the detection module is configured to detect fat thickness and fat density of sites to be detected of a human body;
the determination module is configured to determine body parameters at least based on the fat thickness and the fat density.

2. The body parameter detecting device of claim 1, wherein the detection module includes an ultrasonic sensor configured to detect fat thickness.

3. The body parameter detecting device of claim 2, wherein
the ultrasonic sensor is configured to measure the body sites to be detected ultrasonically to generate a corresponding ultrasonic echo diagram, and determine the fat thickness of the body sites to be detected according to the generated ultrasonic echo diagram.

4. The body parameter detecting device of claim 2, wherein the detection module further includes an electrical conductor sensor configured to detect fat density.

5. The body parameter detecting device of claim 4, wherein the electrical conductor sensor is configured to test electrical conductivity of the body sites to be detected via positive and negative electrodes to obtain currents through the body sites to be detected and determine fat density of the body sites to be detected according to the obtained currents.

6. The body parameter detecting device of claim 1, wherein the determination module is configured to:
consider a set including the fat thickness and the fat density as a current parameter set of the site to be detected; and
determine the obesity index corresponding to the current parameter set of the sites to be detected according to the correspondence relationship between a preset obesity index and a preset parameter set; wherein the preset parameter set comprises a sample fat thickness and fat density of the site to be detected.

7. The body parameter detecting device of claim 1, wherein the detection module further comprises an infrared sensor,
the infrared sensor implements transmission detection on the site to be detected with infrared light, generates a spectral information diagram of the corresponding site to be detected and determines a tissue density of the site to be detected according to the generated spectral information diagram;
the determination module is further configured to determine an obesity index of the corresponding site of a human body according to the fat thickness, fat density and tissue density.

8. The body parameter detecting device of claim 1, further comprising: an output module;
the output module is configured to output the determined body parameters to the user.

9. The fat detecting device of claim 8, wherein the output module is configured to output the determined body parameters to the user via a display module; or
output the determined parameters to the user via a speech output device.

10. A body parameter detecting method, comprising:
detecting fat thickness and fat density of a site to be detected of a human body;
determining body parameters according to the fat thickness and the fat density.

11. The body parameter detecting method of claim 10, wherein the fat thickness of the site to be detected is detected by ultrasonic waves.

12. The body parameter detecting method of claim 11, wherein the site to be detected is measured ultrasonically to generate a corresponding ultrasonic echo diagram, and the fat thickness of the site to be detected of a human body is determined according to the generated ultrasonic echo diagram.

13. The body parameter detecting method of claim 11, wherein the fat density of the site to be detected of a human body is detected by the electrical conductor sensor.

14. The body parameter detecting method of claim 13, wherein the electrical conductivity of the site to be detected of a human body is tested via positive and negative electrodes to obtain a current through the site to be detected of a human body and the fat density of the site to be detected of a human body is determined according to the obtained current.

15. The body parameter detecting method of claim 10, wherein determining body parameters according to the fat thickness and the fat density comprises:
considering a set including the fat thickness and the fat density as a current parameter set of the site to be detected; and
determining the obesity index corresponding to the current parameter set of the site to be detected according to the correspondence relationship between a preset obesity index and a preset parameter set; wherein the preset parameter set comprises a sample fat thickness and fat density of the site to be detected.

16. The body parameter detecting method of claim 10, further comprising:
implementing transmission detection on the site to be detected of a human body with infrared light, generating a spectral information diagram of the corresponding site to be detected and determining a tissue density of the site to be detected according to the generated spectral information diagram; and
determining an obesity index of the corresponding site of a human body according to the fat thickness, fat density and tissue density.

17. The body parameter detecting method of claim 10, further comprising:
outputting the determined body parameters to the user.

18. The body parameter detecting method of claim 17, wherein the determined body parameters are output to the user via a display module or via a speech output device.
